(19) United States
(12) Patent Application Publication
(10) Pub. No.: US 2024/0228934 A9
(43) Pub. Date: Jul. 11, 2024

(54) NONCLINICAL METHOD FOR TESTING MEDICAL DEVICE SURFACE INTERACTIONS WITH MIGRATING CELLS IN SIMULATED IN VIVO ENVIRONMENT

(71) Applicant: CMDC LABS, LONGMONT, CO (US)

(72) Inventors: DANIEL M. STOREY, LONGMONT, CO (US); CHRISTINA K. THOMAS, LONGMONT, CO (US); SIENNA PEARL STOLL, LONGMONT, CO (US)

(21) Appl. No.: 18/490,429

(22) Filed: Oct. 19, 2023

Related U.S. Application Data
(60) Provisional application No. 63/380,159, filed on Oct. 19, 2022.

(51) Int. Cl. C12M 1/00; C12M 1/34; C12M 3/00

(52) U.S. Cl. CPC C12M 29/04; C12M 23/48; C12M 41/00

(57) ABSTRACT

The invention discloses a quasi in-vivo testing method using a uniquely designed multichambered bioblock apparatus. Cells are placed inside a top chamber fitted with a pore size membrane that allows certain cells to migrate through the pores into a bottom chamber holding a substrate. The portion of cells migrating from the top chamber and interacting with the substrate surface in a real time physiological quasi in vivo environment can be used to determine both the migratory response of specific cell types to selected attractive surfaces and the effect of the migratory cells on the surface over selected times of exposure.

### CROSS-REFERENCE TO RELATED APPLICATION

This application claims the benefit of U.S. Provisional Application Ser. No. 63/380,159, filed Oct. 19, 2022, the disclosure of which is hereby incorporated by reference in its entirety.

### BACKGROUND OF THE INVENTION

### Field of the Invention

The present invention relates to an in vitro nonclinical testing method to assess human implant device surface biocompatibility in the bioenvironment to which it is exposed. A specially designed dynamic culture chamber apparatus is used to assess effects in a simulated in vivo environment on the surface of a medical device intended for implantation.

### Description of Background Art

Medical devices include a broad range of products intended for use in the health care system. A medical device as defined by the FDA is an instrument, apparatus, implement, machine, contrivance, implant, in vitro reagent or other similar or related article. Single use, implantable, imaging devices, machines and software can all be used for medical use which may broadly encompass syringes, pacemakers, ultrasound and computer aided diagnostics.

Before a medical device is make available to the public or to health practitioners, its safety in its field of use must be assessed. Safety protocols for all types of medical device used in health care are proscribed in the FD&C Act. The FDA has established medical device classes based on risk posed by the device. Class I is low risk, class II medium risk and class III the highest risk based on safety and any adverse effect on its use in humans. Toothbrushes for example are in class I, pacemakers in class III. Approximately 10% of medical devices are in class III.

Both the United States and the European Union use this classification and base risk assessment of safety on evaluation of possible danger or damage to the human body. Accordingly, a new medical device may not be in use for a long period of time while safety issues are under review by the FDA prior to approval.

The current standard for assessing the interaction of an implanted medical device or biomaterial with a biological system typically consists of performing biocompatibility studies using 2D in vitro cell culture and animal studies. 2D cell-culture followed by testing in animal models is used to simulate the response the human body has to a medical device or biomaterial in contact with tissues and cells. Risk assessments generated from biocompatibility evaluations assist developers in identifying and evaluating potential toxic, physiologic, immunologic or mutagenic effects that material components and their teachables may elicit when used in the human body.

Prior to initiating animal studies, exploratory studies using in vitro benchtop cell culture methods are typically conducted to preliminarily assess biological characteristics of a medical device. According to the FDA, testing risks that can be evaluated in a nonclinical setting or on the benchtop using the device in question in its final finished form should be considered prior to conducting an animal study to support premarket submission. An animal study must be undertaken when the risk analysis study suggests that the safety potential cannot be addressed in an adequate format using non-animal methods.

There are currently no acceptable nonclinical models that address the safety of a medical device used or intended for in vivo use. While the FDA currently supports the “3Rs” principles, to replace, reduce and/or refine animal testing, it is currently digressing from depending on animal models and is focusing on a number of alternatives such as organ-on-a-chip, cell culture and mathematical modeling technologies. Recently, the FDA has begun a pilot program targeting non-animal techniques for testing drugs, biologics and devices. This initiative allows developers to qualify alternative testing techniques that fall outside current evaluation tools such as animal models, biomarker identification and clinical outcome reviews. The openness, growing acceptance and push for alternative testing methodologies from developers and regulatory bodies will be a large step towards improving drugs, biologics and medical devices for humans without harming animals.

Despite efforts from academia, drug and device makers, product manufacturers and regulatory bodies to develop alternative testing methodologies, several technical challenges such as feasibility, qualification and standardization exist regarding the adoption of new technologies that can replace currently authorized techniques. The development of a dynamic in vitro testing model that simulates in vivo conditions would be of value in examining effects of an in vivo environment on medical devices used for implantation.

Two-dimensional cell culture techniques have been traditionally accepted for modeling tissue interactions during in vitro biocompatibility studies. Current technologies, such as 2D culture grow cells in a monolayer on a flat surface and then test in a static environment. Data have shown that 2D cell monolayers have several limitations when predicting how cells behave in a living system, including:

### Feasibility:

2-D cell systems not only are unable to reproduce the elaborate and dynamic environment of in vivo tissue, they also fail to generate accurate results by constraining cell growth and interaction to an artificial, flat or mechanically rigid surface. The spatial organization and cell-to-cell communication capabilities of the cells are severely hindered or eliminated, resulting in a loss of proliferation, differentiation and overall cellular function. The suitability of using 2D techniques for the purposes of evaluating drugs, biologics and medical devices comes into question because of their inability to mimic natural tissue.

### Qualification and Standardization:

Reproducibility, contamination and viability have been reported as issues with 2D cell systems. Reproducibility concerns, in cell culture experiments, result mostly from variation in biological properties between successive passages or generations of cells, variation in culturing parameters, such as soluble gas levels and/or media composition, handling conditions and/or cell line mis-identification. Microbial and impurities are contamination issues.

Recently, in efforts to more effectively mimic the in vivo cell structure not found with 2D monolayer cultures in a manner that can be validated to achieve system specific and reproducible results, approaches are shifting toward three-dimensional (3D) culture systems. Physiological as well as morphological features of tissues can be better restored in a 3D environment by providing cell characteristics and architectures that more closely mimic system-specific models. 3D cell culture could potentially include the introduction of a dynamic system that would affect a local culture environment over the course of time or provide an environment for retaining cell phenotype. A number of 3D cell culture systems or bioreactor technologies are being investigated because of their potential to better mimic tissue-like structures more effectively than monolayer cultures.

### SUMMARY OF THE INVENTION

The disclosed specialized multichamber apparatus places a substrate in an environment that bridges the gap between in vitro and in vivo under simulating physiological conditions. The multichamber system is a novel tool that can reproduce a biological, mechanical or electrical environment in a controlled system. The flexible design of the apparatus enables real time monitoring of cell migration to a substrate surface such as a medical device under quasi in vivo conditions so that reaction of specific cells at a device surface can be tested.

The chamber has a highly adaptable design so that multiple chambers can be integrated with variable 3D environments in a dynamic system. Surface effects from cells migrating to the surface of a substrate located in one chamber can be measured with integrated analytics. The chamber 3D environment can be selected to mimic human serum to test for surface interactions with implanted medical devices. Various cell types that migrate from a fluidly connected first chamber can be in other 3D environments or suitable solvents.

The chamber is uniquely designed so that all testing is performed in real time by utilizing analytic probes attached to the chamber. Testing can be performed at any intervals in the cell migration process without having to interrupt the environment in the testing chamber. This is important especially when long periods of time are needed before seeing any reaction at a substrate surface. The chambers are adaptable for setting up an “in vivo like” environment in which a model medical implant is placed for the tests. A stent for example, would be supported in a chamber with a flow of blood directed through the stent surrounded by endothelial cells. An orthopedic implant can be held between 2 bone like holders, subjected to a very low flow with different types of stem cells and bone forming cells in the solution.

The disclosed chamber apparatus is designed to determine effects of an in vivo environment on safety and performance of an implanted medical device. The use of the novel chamber system to test possible detrimental effects on surfaces of implanted medical devices would require FDA classification as a medical device in class IIb or class III.

### BRIEF DESCRIPTION OF THE DRAWINGS

FIG. 1 shows one version of the disclosed chamber apparatus design in a stacked bioblock test chamber configuration. The stacked bioblock chamber configuration consists of a test chamber 1, an anchor support 2 that holds a substrate positioned above the bottom wall of the test chamber 4, an anchor support port 3, a porous membrane support 12, a porous membrane 13, porous membrane support influx 14 and outflux 15 flow tube ports connected to sterilized male Luer locks with coupler adapters used to connect sterilized tubing, tubing valve flow control devices 11 operating flow between chamber components, adjustable analyte probes 7 and their respective ports 8, adjustable miniaturized microscope probes 5 and their respective ports 6, test chamber influx 9 and outflux 10 ports connected with sterilized male Luer locks with coupler adapters for connecting sterilized tubing; medias/solutions/reagents are transported fluidly throughout the chamber via pumps 16 that are connected to external media/solution/reagent reservoirs 17, vital signs of medias/solutions/reagents are monitored in real time by reservoir analyte/temperature/soluble gas probes 19 that also contain pressure equalizers 18. Soluble gas is delivered to the media/solution/reagent external reservoir via the soluble gas influx flow device 20. Spent media/solution/reagents are transported to external waste reservoirs 21 containing pressure equalizers 18. The test chamber is sealed from the environment with a test chamber blank 22.

FIG. 2 illustrates a sample preparation chamber fluidly connected to the chamber system of FIG. 1. The sample preparation chamber consists of sample preparation chamber A 24 and sample preparation chamber B 25. Media, solutions or reagents enter and exit sample preparation chamber A through the influx 26 and outflux 27 flow tube ports, connected with sterilized male Luer locks with coupler adapters that connect sterilized tubing. Medias/solutions/reagents enter and exit sample preparation chamber B through the influx 28 and outflux 29 flow tube ports, respectively, that are connected with sterilized male Luer locks with coupler adapters used to connect sterilized tubing. Medias/solutions/reagents are transported fluidly throughout the sample preparation chambers via pumps that are connected to external media/solution/reagent reservoirs. The walls of sample preparation chamber A and B including the space between sample preparation chamber A and B are lined with porous filter membranes 30 and the compartment divider 31 connected to the sample preparation chamber via the compartment divider port 32 fluidly isolates contents of sample preparation chambers A and B. The sample preparation chamber is interfaced to the analytical instrument interface 34. Spent media, solution or reagents are transported to external waste reservoirs containing pressure equalizers. The sample preparation chamber is sealed from the environment with a sample preparation chamber blank 33.

### DETAILED DESCRIPTION

The disclosed chamber apparatus utilizes one or more modules that can fit together and engage one another to form a bioreactor and cell culture system for performing 3D cell culture on medical devices. The chamber apparatus can be constructed by assembling one or more individual cell modules together or as a single mold. The modules fit together such that the combined structure encloses a quasi in vivo (QIV) interactive fluid system adapted to measure biological effects and interactions of migrating cells on surfaces in real time.

No current in vitro 3D cell culture technologies for testing the biocompatibility of implanted medical devices are available. Despite 3D and/or bioreactor technologies to evaluate biocompatibility none describe a medical method that uses an adapted human cell bioreactor and cell seeding chamber technology to accurately test medical devices to determine host response and implant integration in the body in an environment that bridges the gap between in vitro and in vivo. The disclosed method of a testing process using the novel chamber apparatus models the host response to an implanted material or medical device. The method is dynamic because it can monitor a time related cascade of biological events such as protein absorption, platelet adhesion and immunological response, tissue restoration and fibrosis.

The quasi in vivo method disclosed herein overcomes major challenges associated with implementing known 3D cell culture techniques. Use of the chamber apparatus as a technology for biocompatibility testing of medical devices provides data on system feasibility, reproducibility, viability and contamination relative to safe use in medical treatment for humans.

In vivo data are important in providing an initial assessment of medical device interaction with a biological system. Tests typically include physiological, pathological and toxicological effects as well as the effect of the biological system on the device. These tests are important when requesting FDA approval for medical devices such as implants.

FDA submissions are ideally supported by in vivo studies. An FDA guidance document is available to help testing facilities as well as persons involved in designing, conducting and reporting results of animal studies intended to assess the safety of devices submitted to the for premarket approval. Following these recommendations facilitates efficient FDA review of medical device submissions that include animal study data.

Although animal studies typically provide initial safety data and may include performance and handling and biological effects when used in vivo, there are no guidelines to proscribe any specific testing related to safety of an implanted medical device. Current review practices are guidelines only published with the intention of promoting consistency.

Testing the evaluable risks on the benchtop to the extent feasible using the device in its finished form can provide safety information. A device in its finished form includes all manufacturing processes for a “to be manufactured” device including packaging and sterilization, if applicable. Before submitting an animal study to support a permanent submission, the FDA recommends completion of nonclinical benchtop performance with the device in its finished form to evaluate potential harms identified with the risk analysis.

The disclosed chamber apparatus has the capability of storing a device/substrate/implant in a dynamic environment that bridges the gap between in vitro and in vivo by simulating physiological, physical and functional conditions in a human patient. The apparatus can be set up to reproduce the biological, mechanical and/or electrical environment of an in vivo environment in a controlled in vitro scenario.

The chamber apparatus comprises a system of one or more bioblocks that can be configured with one another in such a way as to provide an in vivo model by which devices/substrates/implants can be tested for biocompatibility, antifouling, drug dosing and other cell responses and interactions. Such a controlled and reproducible environment simulating in vivo conditions is not possible when performing conventional in vitro testing.

One or more bioblocks can be used to evaluate effects on devices/substrates/implants in a three-dimensional (3D), real-time and high-throughput format. Within each bioblock is a testing chamber with an inner area for insertion of the device/substrate/implant. The testing chamber and its contents are in fluid communication with reservoirs external to the bioblock(s) by way of portals. Each block may contain zero, one or more than one portals and portals may be defined at intakes, outtakes, sampling apertures and support vestibules. Reservoirs external to the bioblocks are fluidly connected by at least one pump by way of the portals and are used to transport fluids, including biofluids such as media, or gasses such as oxygen, carbon dioxide into and out of the testing chambers that house the device/substrate/implant being tested.

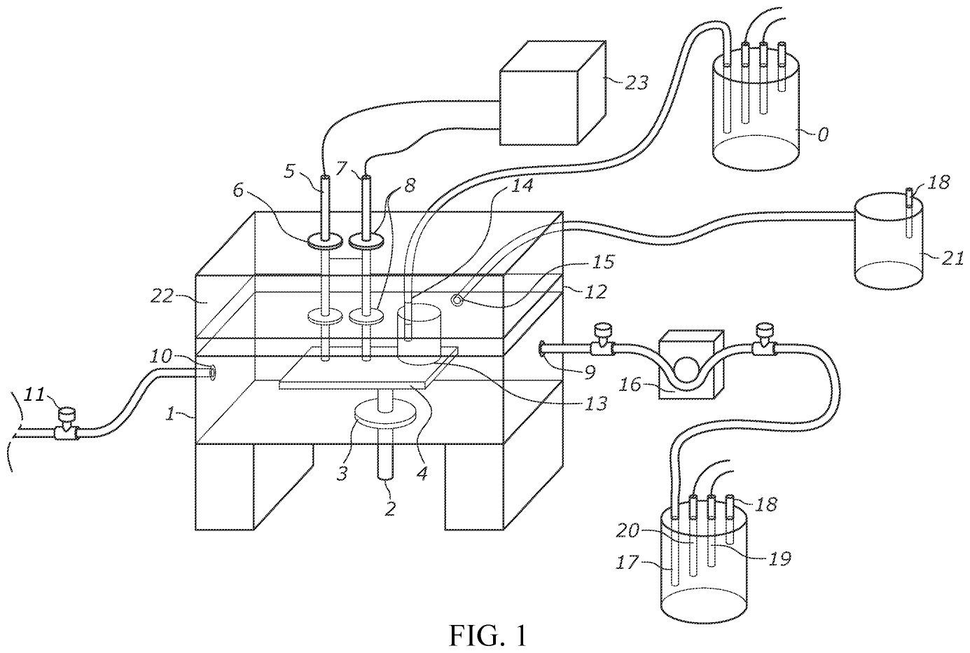

In one example, the chamber apparatus comprises one base bioblock and one stackable bioblock which when engaged with each other in parallel create a construct with testing chambers expandable to retrofit any size and/or shape of device/substrate/implant. Bioblocks engaged with one another allow the testing chambers to be in communication with one another. The base bioblock can contain an intake and an outtake portal that allows fluid to circulate throughout the testing chamber of the base bioblock. An intake portal provides passage of fluid from an external reservoir into a testing chamber and the outtake portal provides passage of fluid out of the testing chamber by a pumping mechanism, see FIG. 1.

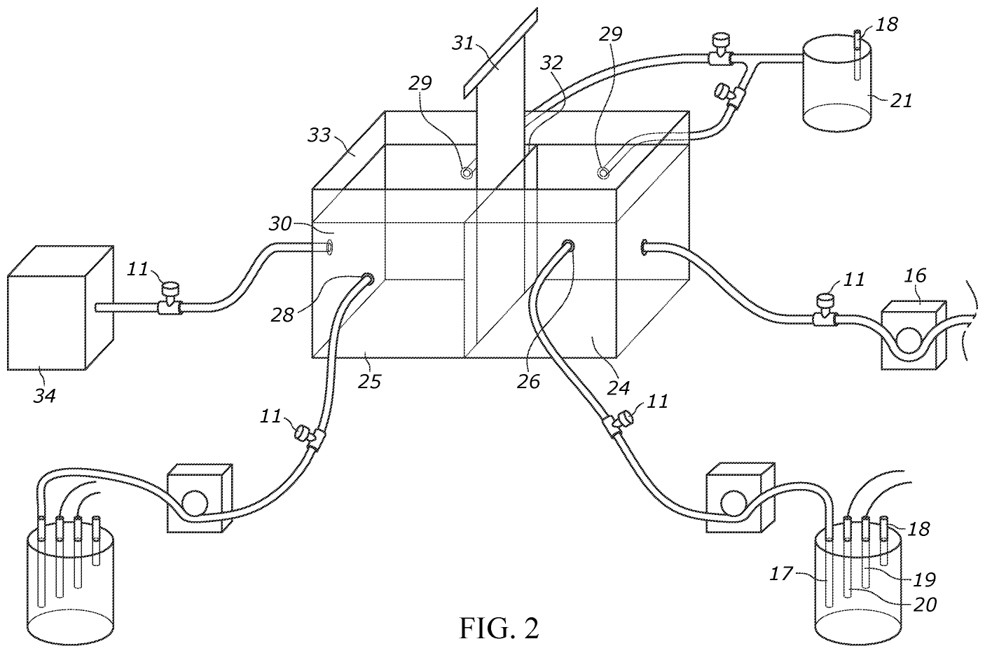

Alternatively, the base bioblock may contain a separate intake and an outtake portal in fluid communication with a testing chamber, FIG. 2. The intake and outtake portals of the base bioblock are moving fluid through a first testing chamber at the same time the intake and outtake portals of the stackable bioblock are moving a different type of fluid through a second testing chamber. In this configuration, fluid from the first testing chamber is in communication with the different fluid from the second testing chamber.

In other configurations, the base bioblock contains an intake portal that allows passage of fluid into the first testing chamber and second testing chamber and passage of fluid through the outtake portal. Both testing chambers share the same fluid with flow being directed into one bioblock and directed out of another bioblock.

A porous membrane is part of the chamber apparatus design and is necessary to separate and control device or substrate surface exposure to migrating cells from a connecting bioblock. The membrane is semipermeable with a pore size selected to allow passage of biomolecules that migrate in vivo to cause effects on the surface of an implanted medical device. Deposited biomolecules on the device might include products from cellular expression from genes, other proteins, small molecules, analytes, cells including mammalian cells, bacteria, virus, tissue particles, waste materials, and liquids/media.

The pore diameter of the membrane is typically 3 to 12 μm in a normal test and is chosen to fit the subject cells. A smaller pore size makes it more difficult for a migrating cell to transverse the membrane. Lymphocytes (10 μm) may go through pores as small as 0.3 μm, whereas the majority of cells, which vary in size from 30 to 50 μm, can move effectively through openings of 3 to 12 μm. Pore diameters of 3 μm are appropriate for testing leukocyte or lymphocyte migration. A fraction of fibroblast cells or cancer cells such as NIH-3T3 and MDA-MAB 231 cells, as well as some immunological cells such as macrophages and monocytes perform most appropriately with a 5 μm pore size diameter. For the majority of cell types, pore sizes of 8 μm are suitable. Most fibroblast and epithelial cells, for example, can migrate most effectively through this pore diameter.

The porous membrane may be any shape or size, for example, in the shape of a flat disc, hollow fiber or tubular and may be flexible, semi-flexible or rigid. Porous membranes can be stacked such that an outer membrane carrier can exchange entities to an inner membrane. Porous membranes work by delivering exchange entities by diffusion, bulk flow or any other transport mechanism. The pore size selected will typically be between 0.1 and 20 μm but may also be in the nanofiltration or ultrafiltration range according to a particular application.

Porous membrane material can be made of one or more or combination of any hydrophilic or hydrophobic materials. The porous membranes may consist of one or more layers made up of woven or nonwoven fibers or material sintered from particles or one or more or combinations of co-polymers of natural or synthetic polymers or inorganic material. Examples include aliphatic polyamides, polysulfones, polyethersulfones, polyesters, polyvinylidene halides, acrylic polymers, acrylic copolymers, cellulose esters, polyalkenes and halogenated polyalkenes. The porous membrane is any material that may come into contact with cells, nutrients, growth factors, gel matrices, fluids or other biochemicals without causing an adverse reaction. The materials should be suitable for sterilization and be biocompatible. The surfaces may be modified for the purposes of encouraging or discouraging cell attachment. The membrane may also consist of macro-/micro-/nano-particles.

At least one porous membrane is comprised within the disclosed chamber apparatus which can be oriented between adjoining bioblocks or fixed between bioblocks. A support or scaffolding properly orients the porous membrane such that it can be positioned horizontally or at any position or depth within a bioblock; for example, between the portals of the bioblocks. The porous membrane can be oriented vertically or at any angle and be located at any position within a bioblock. The porous membrane is preferably semipermeable and may expand a distance of more than one bioblock. Some membranes may be selected to allow passage of a permeable gas such as nitrogen, carbon dioxide or oxygen produced from cellular expression. Custom membranes may be used to direct permeability of other materials in the bioblock testing chamber.

A porous membrane support is considered to be a chamber bioblock that fits together or engages with other bioblocks including a test chamber to form a bioreactor and cell culture system for performing 3D cell culture on medical devices. One or more porous membrane supports may be stacked on top of one another in a combined structure or multiple porous membrane supports may be constructed in a single mold. Porous membrane supports may also be constructed in a single mold with test chamber(s) or any other component of the chamber apparatus. Porous membrane support bioblocks together or with other chamber component bioblocks are engaged, locked, sealed together with one another by any mechanism that prevents leakage from the chamber and maintains a sterile environment. The sealing mechanism may be temporary for example with O-ring or compression fitting or permanently fixed with glue or sealant. The porous membrane bioblocks may be fixed together or be fixed with other components of chamber with endcaps fastened to the face of the chamber using washers, bolt, O-rings. The endcaps are held against the chamber face using threaded lines that align and connect the test chamber bioblocks and/or other components of the chamber.

Each porous membrane support bioblock contains at least one porous membrane. When multiple membranes are used, the porous membrane support bioblock will contain multiple, separate compartments isolated from one another via non-porous walls in the porous membrane support block. A single compartment may contain several stacked membranes. Multiple compartments in the porous membrane support bioblock may be utilized if the migratory effect of more than one cell type is to be assessed. A stacked porous membrane support system can be utilized to recreate a neuronal tissue system on a medical device in a complex system such as for the retina of eye. Such an arrangement could be used to evaluate the reaction of photoreceptors, bipolar cells, retinal ganglion cells, horizontal cells and amacrine cells in an anatomically correct orientation in reaction to the medical device.

The porous membranes can be secured to or in the porous membrane support bioblock via a clamping mechanism, by a mechanism that includes the porous membrane as part of the mold. The porous membrane support bioblock can comprise individual ports for flowing media/solution/reagent in or out, for sampling, for diagnosing analyte or gas concentration or temperature. The porous membrane support bioblock can be of any size and geometry as can the separate compartments that house the porous membranes.

The porous membrane support bioblock may be made of plastic, glass, metal, quartz, other ceramic or moldable or formable material that will come into contact with cells, nutrients, growth factors, gel matrices, fluids or other biochemicals in the chamber without causing an adverse reaction. The support material should be suitable for sterilization and be biocompatible. The surfaces can be modified to encourage or discourage cell attachment. Surface material may include stainless steel, silicone, Duran glass, low-density polyethylene, silicone, Viton or any material that is inert, and suitable for cell culture; or suitable for disposable purposes of reusable; or any suitable material for sterilization/resterilization.

A porous membrane can be oriented between adjoining bioblock components fixed between bioblock components with a support. A support orients the porous membrane so that it can be positioned horizontally at any position or depth within the chamber; for example, between the portals of the bioblock components. It may be configured such that the porous membrane is oriented vertically or at any angle and position within a chamber. The porous membrane may expand a distance of more than one bioblock.

The porous membrane allows cells, analytes, gas, liquid, to exchange in the chamber. The porous membrane is semipermeable with any pore size and allows passage of permeable gases such as nitrogen, carbon dioxide and oxygen, molecules produced from cellular expression, genes, proteins, small molecules, analytes, cells including mammalian cells, bacteria, virus, other particles, waste materials, or liquids/media. The porous membrane may be any shape or size. For example, the porous membrane may be in the shape of a flat disc, hollow fiber or tubular and may be flexible, semi-flexible or rigid. The membranes may be stacked such that, for example, an outer membrane carrier exchange entities to an inner membrane. The porous membranes may work by delivering exchange entities by diffusion, bulk flow or other transport mechanism. The pore size may be between 0.1 and 20 μm and may be selected according to application. The pore size may also be in the nanofiltration or ultrafiltration range.

A bioblock is typically constructed to contain a scaffold or support to secure an implant device during exposure to a synthetic in vivo environment. One or more supports can be located within the culture chamber of the module and used to hold or fix the device/substrate being tested in place within the culture chamber. The support can act as a direct or indirect interface between the tissue and the implant/implant surface. Substrate support refers to the one or more framework structures on which or wherein a device/substrate can be placed on or attached to that can provide lateral or vertical support. The support can also be utilized to simulate movement and provide external stimuli to the device/substrate in the culture chamber.

The support material for a substrate in a test chamber can be natural or synthetic and mimic the naturally occurring tissue and structure that a device/substrate being tested will be in contact with or attached to. Supports can be made of any number of materials including metals, plastics, and ceramics and selected to be used under particular conditions such as thermoplastic polymer scaffolds. One support may directly support the device/substrate or support may be provided by one or more abutments. Types of abutments built into the chamber can include stock abutments, tissue level, straight, angled, prep-able, bone level, screw retained, custom abutments, milled, UCLA (University of California Los Angeles Abutment), locator, or cemented.

Substrate supports may simulate function using dynamic loading on the device/substrate being tested. The support itself can mimic the native structure of an implantation site. For example, an osseous tissue device/substrate/implant placed in the cell culture chamber may be surrounded or enveloped directly by a support that includes both hard and/or soft tissue and material mimics such as synthetic bone graft material, including beta-tricalcium phosphate, autogenous bone and resorbable collagen membranes. A device/substrate/implant may be fixed/suspended in the cell culture chamber with surgical pins with or without the hard and/or soft tissue and material mimics.

Supports may be constructed from materials with a wide range of chemical, physical or mechanical properties and may range from a hydrogel to a metal. Support material may be a synthetic or natural biodegradable non-biodegradable co-polymer or mixture thereof. The matrix or environment may include one or more of the following materials: any polylactide, chondroitin sulfate, polyester, polyethylene glycol, polycarbonate, polyvinyl alcohol, poly-s-caprolactone.

Materials used for hard supports may include materials such as tissues and for soft tissue mimics may include hydrogels which may consist of one or more of any of the following homopolymers, copolymers (triblock, multiblock) and interpenetrating polymers: nonionic, cationic, anionic, ampholytic, biodegradable, non-biodegradable, physically crosslinked, chemically crosslinked, smart, conventional, natural, hybrid and/or synthetic.

Supports may be manufactured or molded as part of the bioblock so that the bioblock and the support are one continuous structure. The supports may be separate structures attached or assembled to the bioblock by means of a clip, Luer lock, screw, cement, or similar fastener.

During dynamic testing in a bioblock, a medical device located within the chamber can be exposed to one stimulus or may be exposed to one or more electrical, mechanical, chemical, functional and/or physical events by incorporating a mechanism for providing stimulation to the cells and/or medical device positioned within the bioblock. By applying stimuli to cells, to their scaffold and/or a medical device, cell growth, orientation and remodeling can be manipulated.

The bioblocks can also be used to grow and isolate different types of cells for different applications. The porous membranes, oriented in between each bioblock containing a particular cell type can selectively allow passage of products of cellular expression such as proteins from cells in one bioblock to cells in a second bioblock. For example, cells in one bioblock can be stimulated to release any number and type of soluble factors like cytokines, chemokines. The released soluble factors transport through the porous membrane into a second bioblock containing undifferentiated cells. Transport of the soluble factors into the second bioblock and subsequent exposure of the undifferentiated cells to the soluble factors released from the cells in bioblock one can comprise a response such as triggering the undifferentiated cells in the second bioblock to differentiate.

The design of the multichamber apparatus is flexible and can be modified to provide defined environments for a particular medical implant. The design includes ports which are important in creating a real time interactive environment with the surface of the medical implant housed in one of the interconnected chambers.

The ports provide fluid communication between the internal culture chamber volume and the external environment. The ports facilitate flow between modules that are in series. In such a scenario the ports allow fluid communication between the internal culture chamber volume of one module with another module. The ports may include inlets, outlets and sampling ports. The ports can provide flow into and/or out of each individual module. When flow occurs independently through each module, a unique culture environment can be created and controlled within each module. If there are three individual modules engaged with one another, each module containing respective inlet, outlet and sampling ports, can be controlled individually to create up to three separate culture environments in one chamber apparatus with the three modules can working in unison to create one culture environment.

Ports can be selected for each module such that inlet, outlet and sampling ports exist on independent modules. For example, the flow inlet may be located on one module, the flow outlet may be located on a second module and the sampling port may be located on a separate module. Other configurations of inlet, outlet and sampling ports may be envisioned. Ports may be located on any face/plane of the module. They may extend outwards or protrude externally from the surface of module, as shown in 26, FIG. 2, or may extend inwards or protrude internally from the surface of the module, as shown in 29, FIG. 2, or may be flush with the wall of a module, as shown in 9 and 10, FIG. 1, such that they do not protrude externally or internally.

The ports may be any shape or size. They may be symmetrical or asymmetrical. They may contain any cross-sectional geometry of any size. Cross-sectional shapes may include, but are not limited to, circle, semicircle, oval, triangular, square, rectangle, parallelogram, rhombus, trapezium, kit, pentagon, hexagon, heptagon, octagon, nonagon, and decagon. The flow ports are strategically placed throughout the chamber apparatus in order to achieve the desired flow characteristics. One or more ports can be arranged at any location around the module. They may be arranged in equidistant or random patterns to provide the desired perfusion of media or fluid throughout the culture chamber and around the medical device being evaluated. Different combinations of ports with different locations, sizes and geometries of ports may be utilized to achieve flow parameters such as magnitude and direction of flow that allow perfusion to occur more efficiently around small and narrow area of a medical device.

Taking advantage of different port locations, sizes and geometries of the ports provides a more accurate representation of fluid flow in a 3D in vitro-like environment. Flow characteristics achieved through selective placement of inlet and outlet ports will promote transport of nutrients and gases such as O₂ or CO₂, removal of cellular waste, other nutrients or biomolecules vital for mimicking healthy cellular growth in a 3D in vivo-like environment. Ports can also be used to deliver cells into the culture chamber for cell seeding.

Ports can also be used for connecting physiological sensors, such as pH or dissolved gasses to the inner environment of the culture chamber so that real-time measurements or system adjustments can be made. Medical devices and substrates located in the chamber apparatus can be cultured in a 3D in vivo-like environment. Ports may be used to deliver cells into the chamber as well as collect cells as they are released from the chamber or the scaffolding material of the chamber. Ports may be used to collect cells or media for analysis. Ports are used to deliver or collect media, cells so that the system/culture is not disturbed. Ports can also be used to deliver other materials to the chamber. Other material can be chemical signaling agents such as cytokines, growth factors and chemokines. Ports can be opened or closed using any method known to one in the art such as clamps or valves or may be controlled by pumps.

One or more supports located within the culture chamber of the module can be used to hold or fix the device/substrate being tested in place within the culture chamber. The supports act as a direct or indirect interface between the tissue and the implant/implant surface. A support is one or more framework structures on which a device/substrate can be placed on or attached to and which will provide lateral or vertical support or resist movement. Supports can also be utilized to simulate movement. The support can be natural or synthetic and can mimic the naturally occurring tissue and structure that a device/substrate being tested will be attached to. Supports can be made of any number of materials including metals, plastics, and ceramics. The supports may directly support the device/substrate or the device/substrate being tested may be supported by one or more abutments. The supports may simulate function by placing dynamic loading on the device/substrate being tested.

The support can mimic the native structure of the implantation site. An osseous tissue device/substrate/implant in the cell culture chamber may be surrounded or enveloped directly by a support that includes both hard and/or soft tissue and material mimics such as synthetic bone graft material, including but not limited to beta-tricalcium phosphate, autogenous bone and resorbable collagen membranes. An osseous tissue device/substrate/implant may be fixed/suspended in the cell culture chamber with surgical pins with or without the hard and/or soft tissue and material mimics. The materials from which the supports are constructed may have a wide range of chemical, physical and mechanical properties.

### Definitions

As used herein:

Bioblock—A controlled environment or apparatus designed for studying, examining, or testing biological samples, such as cells, tissues, or microorganisms, under specific conditions. Bioblock chambers can hold complex systems that stimulate body or environmental conditions.

MBA refers to the disclosed multichambered bioblock apparatus specially designed for use with a QIV environment to test cell migration onto medical device surfaces in real time conditions.

3D environment—This environment provides a spatially three-dimensional matrix or scaffold where cells or tissues can grow in all directions (x, y, and z axes). This allows cells to interact with their surroundings and with other cells in a manner that more closely resembles their natural, in vivo conditions.

Quasi in vivo or QIV—a condition or system that stimulates or comes close to the conditions of a living organism but is not truly within a living entity. This refers to a model or experimental system that attempts to closely mimic the in vivo environment without actually being in a living organism.

Migrating cells—cells that will transverse a semipermeable membrane due to extracellular signaling, but in the absence of any signaling, will not transverse the semipermeable membrane.

Biofilm—A complex assembly of microorganisms, such as bacteria, fungi, an algae, that adhere to surfaces and are embedded within a protective matrix of self-produced extracellular polymeric substances (EPS). Biofilms can form on various surfaces and are often resistant to antimicrobial agents.

### Materials

S. aureus cultures were obtained from ATCC, Manassas, VA.

Tryptic soy broth was purchased from Sigma Aldrich, St. Louis MO.

RPMI 1640 was purchased from Gibco BRL, Grand Island, NY.

5% fetal bovine serum, heat inactivated, and SytoBC™ were purchased from Life Technologies, Carlsbad, CA.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLES

### Example 1: Biofilm Formation on a Substrate in a Quasi In Vivo Environment

The test chamber apparatus design used, see FIG. 1, is a system of one or more interlocked bioblocks used to maintain cells in a closed control environment that models an in vivo system. In this example, the top chamber or porous membrane support 12 contains a cultured cell solution separated by a porous membrane 13 from the bottom chamber 1 which contains a test substrate 4. Effect of cells migrating cells through the porous membrane 13 from the top chamber or porous membrane support 12 onto the test substrate 4 over time was assessed by flowing cell media through chamber 1 via intake 9 and exit 10 outflow and exit ports.

Titanium coated PEEK and uncoated PEEK substrates were each fixed, in independent test chambers, with an anchor support that holds the substrate securely in place, centered, approximately in a position paralleling the bottom wall of the bioblock. The surface of the substrate is positioned 100 μm from the porous membrane and the tips of the adjustable miniature analyte probes and adjustable miniature microscope probes.

A bioblock chamber apparatus similar to FIG. 1 was used to form a biofilm on a test substrate. A 1×10⁴ cells/ml solution of S. aureus in tryptic soy broth was placed in the external solution reservoir 7 connected fluidly to chamber 1. Inlet and outlet tubing valve devices 11 were used to control flow the of S. aureus solution via pump 16 to chamber 1. Either an uncoated PEEK or titanium coated PEEK substrate 4 was attached to a support 3 in chamber 1 and was exposed to the S. aureus solution for 14 days at room temperature under static or flow conditions of 0.1 ml/min.

Visual examination of biofilm stained with nucleic acid dye SytoBC™ was used to compare biofilm growth under static and dynamic flow conditions. No biofilm formed on the titanium coated PEEK under static or flow conditions after 14 days. Biofilm was observed on uncoated PEEK under both static and positive flow conditions after a few days and was well established after 14 days.

### Example 2: Preparation of PMN Cells

A PMN cell suspension was prepared by isolating cell from human whole peripheral blood with EDTA Anticoagulant using an EasySep® Direct Human Pan-Granulocyte Isolation Kit according to the manufacturer's instructions. After isolation, unactivated PMNs were resuspended in a complete media consisting of RPMI 1640 containing 5% Fetal Bovine Serum, Heat Inactivated, and 120 U/L Gentamicin. PMN cells were labelled with a live cell CellTracker Green™ CMFDA Dye according to manufacturer's protocol. Following isolation and labelling the PMN cells were transferred to an external test chamber media/solution/reagent reservoir 7 with gentle agitation to prevent cells from adhering to the surface of the reservoir.

### Example 3: Polymorphonuclear Cell (PMN) Effect on Biofilm Coated Surface

This example describes use of the chamber apparatus in FIG. 1 to measure interaction of PMN cells, including neutrophils, eosinophils, basophils and mast cells, with a Staphylococcus aureus biofilm grown on a substrate surface using a simulated in vivo environment intended to resemble a typical inflammatory response to a biofilm coating on an implanted medical device.

A S. aureus biofilm was grown for 14 days on a titanium-coated or uncoated PEEK substrate 4 as described in Example 1. A 1×10⁵ cells/ml PMN suspension was prepared as described in Example 2 and injected via an inlet porous membrane support influx flow tube port 14 into the porous membrane support 12 holding an 8 μm pore diameter, 200 μm thick polycarbonate membrane 13. The PMN cell suspension was prepared in 2 ml of RPMI 1640 complete media conditioned with CO₂ and O₂ to generate a solution pH between 7.0 and 7.4. The inlet and outlet tubing valve control devices 11 were opened and media containing PMNs allowed to flow into the porous membrane support 12 via pump 16. Once full, the inlet and outlet tubing valve flow control devices 11 were closed and the pump 16 stopped.

Chamber 1 was filled with RPMI 1640 media to fully immerse a titanium coated or uncoated PEEK substrate 4. Under static flow conditions, the flow of RPMI 1640 complete media to the test chamber 1 was discontinued by shutting off flow via the inlet and outlet tubing valve flow control devices 11. Under dynamic conditions, the flow of RMPI 1640 during the experiment was continuous at 0.1 ml/min. Immediately after immersing the substrate 4 in RPMI media, the PMN solution was injected to volume in the porous membrane support 12.

The number of PMN cells adhered to the surface of the substrate in the test chamber 1, was counted after 24 hours using the miniature microscope probe 5. PMNs were observed on surfaces of PEEK that contained S. aureus biofilms. Under static conditions, a slightly higher number of PMNs were visibly adhered to the uncoated PEEK surface already coated with biofilm. Few or no PMNs were observed on surfaces without any biofilm or titanium coated PEEK under both dynamic and static conditions.

### Example 4: PMN Phagocytosis

PMN phagocytosis is tested under static and dynamic conditions as described in reference FIG. 1 and FIG. 2. Staphylococcus aureus biofilm is grown on titanium coated PEEK and uncoated PEEK for 14 days and labeled with a nucleic acid dye, as described in example 1. After staining, phosphate buffered saline is pumped through the test chamber 1 to remove residual, unreacted SytoBC™. The inlet and outlet tubing valve flow control devices 11 are opened, and PBS is allowed to flow into and out of the test chamber via the inlet port 9 and outlet ports 10, respectively, of the test chambers 1 that are connected with sterilized male Luer locks with coupler adapters used to connect the sterilized tubing to an external solution reservoir containing PBS. Unused SytoBC™ is simultaneously pumped into the external waste reservoir 21. Once reagent exchange is complete, the inlet and outlet tubing valve flow control devices 11 are closed and the pump 16 stopped. Both reservoirs are pressure equalized.

After 14 days of biofilm formation on the PEEK substrate, a suspension of PMNs (1×10⁵ cells/mL) is injected into the porous membrane support 12, which is outfitted with an 8 μm pore diameter, 200 μm thick polycarbonate membrane 13, via the inlet port 9 of the porous membrane support 12 that is connected with a sterilized male Luer lock with coupler adapters used to connect the sterilized tubing to an external solution reservoir 7 containing a suspension of PMNs in RPMI 1640 complete media that is that is conditioned with sterile-filtered CO₂ and O₂ to generate a solution pH of between 7.0 and 7.4. The inlet tubing valve flow control device is opened and 2 ml of media containing PMNs is allowed to flow into the porous membrane support 12 via peristaltic pump 16. Once full, the inlet tubing valve flow control device 11 is closed and the pump is stopped.

The test chamber 1 is filled with RPMI 1640 media fully immersing the titanium coated or uncoated Peek substrate 4. Under static flow conditions, the flow of RPMI 1640 complete media to the test chamber 1 is discontinued by shutting off flow via the inlet and outlet tubing valve flow control devices 11 and pump 16. Under dynamic conditions, the flow of RPMI 1640 is continuous at 0.1 mL/minute. Immediately after immersing the substrate 4 in RPMI 1640 media, the PMN solution is injected to volume in the porous membrane support 12.

PMN cells are incubated for 1 hour at 37° C. and at 4° C. (as a control) to allow chemotactic migration through the porous membrane 13 into the test chamber 1 onto the substrate 4, and then and then trypsinized from the surface using a Trypsin/EDTA in PBS solution injected into the test chamber via the inlet port 9 of the test chamber 1 that is connected by sterilized tubing to an external media/solution/reagent reservoir 17 containing the Trypsin/EDTA in PBS solution. Following trypsinization, the tubing valve control devices 11 between the test chamber 1 and the sample preparation chamber 24 are opened and the trypsinized cell solution is transferred from the test chamber into the sample preparation chamber via pump 16. The trypsinized cell suspension enters the sample preparation chamber via sterilized tubing that is connected to the sample preparation chamber. In sample preparation chamber 24, the PMN cells are pelletized and the Trypsin/EDTA is replaced in volume with PBS containing 1% (w/v) paraformaldehyde. 1% (w/v) paraformaldehyde solution is simultaneously pumped into the sample preparation chamber as spent Trypsin/EDTA solution is being pumped into the external waste reservoir.

Once reagent exchange is complete, the inlet and outlet tubing valve flow control devices 11 are closed and the pump 16 is stopped. Both reservoirs are pressure equalized. The adjustable compartment divider 31 is then opened and the cells transport from sample preparation chamber 24 to sample preparation chamber 25. A 5 mM solution of crystal violet in 0.1M sodium chloride is injected via the inlet port 28 into the sample preparation chamber 25. Sterile tubing connects the chamber to an external reservoir 17 containing the crystal violet solution. The tubing valve control device 11 between the sample chamber 25 and the external reservoir is opened and the crystal violet solution transferred into the sample preparation chamber 25 via pump 16. Once transfer is complete, the tubing valve control device 11 is closed and the pump stopped. After incubating for 45 minutes at room temperature, the tubing valve flow control device connecting the sample preparation chamber 25 and the flow cytometer analytical instrument interface 34 is opened and PMN solution is pumped into the flow cytometer to be analyzed for fluorescent intensity (gated for PMNs carrying SytoBC′).

### Example 5: Interaction of Bone Marrow Derived Mesenchymal Stem Cells (Bm-MSC) with a Titanium Coated Versus Uncoated Orthopedic Spinal Implant

The multichamber system shown FIG. 1 and FIG. 2 was used in the stacked chamber configuration. Titanium coated and uncoated PEEK orthopedic spinal implants are each fixed in independent test chambers 1 using an anchor support 2 to hold the implant centered securely in place approximately perpendicular to the bottom wall of the test chamber. The surface of the PEEK orthopedic spinal implants is positioned 100 μm from the tips of the adjustable miniature analyte probes 7 and adjustable miniature microscope probes 5.

A bm-MSC cell suspension in Mesenchymal stem cell basal medium for bone-marrow derived MSCs is prepared and the cells cultured through several passages. After pelleting and collection, the cells are resuspended in complete media at a density of 2×10⁴ cells/ml. Before use, the cells are labeled during culture in order to track during test procedures in the chamber system.

The cell suspension is transferred to an external solution reservoir 17 and maintained at constant temperature and humidity. The titanium coated and uncoated PEEK orthopedic spinal implants 4 are primed in the test chamber by injecting complete medic into the test chamber 1 via the test chamber influx flow tube port 9 that is connected by a sterilized male Luer lock with coupled adapters to an external media reservoir 17 containing complete media maintained at 37° C., 5% CO², 95% humidity for culturing. The inlet tubing valve flow control device 11, is opened to moderate the flow of complete media into the test chamber 1 completely surrounding the orthopedic spinal implants. Once immersed, the inlet tubing valve flow control devices 11 are closed and the peristaltic pump 16 is stopped to halt the media flow.

A suspension of bm-MSCs (2×10⁴ cells/mL) is injected into the test chambers 1 containing the titanium coated and uncoated PEEK orthopedic spinal implants 4 via the test chamber influx flow tube port 9.

The inlet and outlet tubing valve control devices 11, are opened and the volume of complete media in the test chamber 1 is replaced with bm-MSC cell suspension via peristaltic pump 16 at a rate of 0.1 mL/minute. After the cell suspension transfer is complete, the inlet and outlet tubing valve flow control devices 11 are closed and the flow is stopped to allow bm-MSC seeding on the orthopedic spinal implant 4 surface. The test chamber is maintained under appropriate culturing conditions.

The bm-MSCs are cultured on the titanium coated and uncoated PEEK orthopedic spinal implants under dynamic flow conditions for 14 days. Under dynamic conditions, the flow of complete media during the experiment is continuous at 0.1 mL/minute over the desired time period. The proliferation of bm-MSC cells is assessed in without opening the test chamber 1 and/or without substrate/cell destruction by counting cells in 10 random spots on the substrate surface using the adjustable miniature fluorescent microscope probe 5.

Dynamic flow in the test chamber is established by injecting complete media into the test chamber containing the titanium coated and uncoated PEEK orthopedic spinal implants. The inlet and outlet tubing valve flow control devices moderating the flow of complete media, are opened and the volume of complete media evacuated from the test chamber into the external waste reservoir 21. Media is continually being replaced with fresh complete media via peristaltic pump. After 7 days bm-MSC cells are trypsinized off the surface of the titanium coated and uncoated PEEK orthopedic spinal implants using a trypsin/EDTA in PBS solution injected into the test chamber. The inlet and outlet tubing valve control devices moderating the flow of the trypsin solution are opened and the spent complete media removed from the test chamber into the external waste reservoir 21 while being continually replaced with the trypsin solution via the pump 16. Following media replacement, the flow devices are closed and the pump is stopped. The test chamber is incubated for 15 min.

Following trypsinization, the tubing valve flow control devices between the test chamber and the sample preparation chamber 24 are opened and 1 mL of the trypsinized cell solution is transferred into the sample preparation chamber. The trypsinized cell suspension enters the sample preparation chamber A (24) via sterilized tubing connected to the sample preparation chamber. Following solution transfer, the tubing valve flow control devices 11 are closed and the pump stopped.

In sample preparation chamber, the bm-MSC cells are pelletized and the Trypsin/EDTA in PBS solution is replaced in volume with fresh complete media. Complete media is simultaneously pumped into the sample preparation chamber as spent Trypsin/EDTA solution is being pumped out to the external waste reservoir 21 via the outflux flow tube port 27. Solution exchange occurs when the tubing valve flow control devices between the sample preparation chamber and the external media reservoir 17 and the sample preparation chamber and the external waste reservoir 21 are opened and spent Trypsin/EDTA solution is replaced with fresh complete media. Once reagent exchange is complete, the inlet and outlet tubing valve flow control devices are closed and the peristaltic pump is stopped. All external reservoirs are pressure equalized via pressure equalizers 18.

Pelletization of the bm-MSC cells is repeated and the complete media is replaced in volume with PBS solution. Once reagent exchange is complete, the inlet and outlet tubing valve flow control devices are closed and the peristaltic pump is stopped. All external reservoirs are pressure equalized via pressure equalizers.

The bm-MSC cells again are pelletized and the spent PBS solution in the sample preparation chamber is removed using a syringe that is interfaced at the sample preparation chamber outflux flow tube port 27. SingleShot® cell lysis master mix, is injected into the sample preparation chamber via influx flow tube port 26 that connects sterilized tubing to an external solution reservoir 17 containing the SingleShot® cell lysis master mix solution. Solution transfer occurs when the tubing valve flow control device 11 between the sample preparation chamber and the external solution reservoir is opened. Once transfer is complete, the tubing valve flow control device is closed and the peristaltic pump is stopped. All external reservoirs are pressure equalized via pressure equalizers 18. After resuspension, the cells in the sample preparation chamber are incubated.

After appropriate incubation time, the adjustable non-permeable sample preparation chamber divider 31 is opened and the cell suspension is transported from sample preparation chamber 24 through sample preparation chamber 5 directly into a nucleic acid purification station and RT-PCR analytical instrument interface 34. Within the analytical instrument interface 34 bm-MSC lysis solution is processed for RNA purification and then RT-PCR. Once transfer is complete, samples of media could be aliquoted for RT-PCR testing gene expressed proteins. Once the samples are removed, the tubing valve flow control device is closed and the peristaltic pump stopped.

Samples aliquoted from the sample preparation chamber 5 are tested by real-time polymerase chain reaction (RT-PCR) at day 7 under dynamic conditions for intracellular alkaline phosphatase (ALP). The amount of intracellular alkaline phosphatase in the media is assessed at periodic times during dynamic flow conditions during which time the bm-MSCs are cultured on the titanium coated and uncoated PEEK orthopedic spinal implants.

Results show that cell adhesion occurred on titanium coated and uncoated PEEK orthopedic spinal implants; however the number of cells adhered to the coated surface after one hour is greater that the number of cells on the uncoated PEEK orthopedic spinal implant. Additionally, the titanium coated PEEK orthopedic spinal implants demonstrate enhanced cell proliferation over the course experiments compared to the uncoated PEEK orthopedic spinal implant. Cells reach their proliferative capacity on the titanium coated PEEK orthopedic spinal implants around day 7 whereas the uncoated PEEK orthopedic spinal implants lag behind, reaching a plateau at day 14. A similar trend for gene expression is seen between the titanium coated and uncoated PEEK orthopedic spinal implant surfaces. The coated surface is expected to display enhanced synthesis of osteogenic genes (ALP, BMP2, RUNX2, COL1, OPN and OCN) as well as calcium at 7 days compared to the uncoated surface.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

We claim:

1. A multichamber bioblock apparatus (MBA) for testing migratory cell effect on a medical device surface in a biosimulated in vivo environment; comprising,
a cell bioreactor chamber;
a cell seeding chamber separated by a porous membrane from the cell bioreactor chamber;
a support in the cell bioreactor chamber for attaching a model medical device;
inlet and outlet flow tube ports on the bioreactor chamber;
an external media reservoir equipped with a pump;
one or more multiparameter analysis probes interfaced with the chambers;
wherein a dynamic cell culture suspension in the external media reservoir is pumped into the bioreactor or cell seeding chamber for time periods that allow cells to migrate from the cell suspension in the one bioreactor chamber through the porous membrane into the other chamber from which changes over time at the medical device surface caused by migrating cells are measured with an analysis probe.

2. The MBA of claim 1 wherein the porous membrane has a pore size to passage selected cell types from the cell seeding chamber into the fluidly connected cell bioreactor chamber.

3. The MBA of claim 2 wherein the selected cell types are fibroblast, macrophage, epithelial, or lymphocyte cells.

4. The MBA of claim 1 wherein a cell suspension of S. aureus cells from the bioreactor chamber pass through the porous membrane into the cell seeding chamber.

5. The MBA of claim 1 wherein S. aureus cells from the cell suspension pumped in from the external reservoir form a biofilm on the surface of the medical device after 14 days.

6. A nonclinical evaluation method of testing in vivo biocompatibility risk of a medical device intended for implantation in humans, the method comprising:
a) supporting a model medical device in a first chamber of a multiunit interlocked bioblock chamber system containing a physiological environment;
b) providing an active cell suspension via influx and outflux port tubes from an external reservoir into a second bioblock chamber separated from the first chamber by a porous membrane;
c) flowing the active cell suspension from the external reservoir into the second bioblock;
d) measuring time dependent changes in the cell suspension exiting from the second bioblock chamber port;
e) assessing effect from cells migrating through the porous membrane onto the device surface over selected time periods of flow; and
f) relating effects of migrating cells on the device surface to safety or biocompatibility risk for use in vivo.

7. The method of claim 6 wherein the cell suspension contains fibroblast, macrophage, epithelial, or granulocytes.

8. The method of claim 6 wherein migrating cells onto the device surface form a biofilm.

9. The method of claim 6 wherein the pore size of the membrane is 3-12 μm.

10. The method of claim 6 wherein the physiological environment in the first chamber is compatible with human cells.

11. The method of claim 6 wherein time period of flowing is 14 days.

12. The method of claim 6 wherein flowing has a flow rate in the range of 0.1 μm/min.

13. The method of claim 6 wherein analytical probe ports are connected to the first and second bioblock chambers.

14. A quasi in vivo (QIV) test performed in the multichamber bioblock apparatus of claim 1 on a medical device intended for implantation supported in a human compatible tissue environment, wherein the test data are used to compare changes on the medical device surface over time to other medical device surfaces that are unaffected in the same test conditions.
